(19) United States
(12) Patent Application Publication
(10) Pub. No.: US 2023/0279493 A1
(43) Pub. Date: Sep. 7, 2023

(54) TESTING ASSAY FOR SCREENING AND DIAGNOSIS OF USHER, PENDRED, JERVELL, AND LANGE-NIELSEN SYNDROMES

(71) Applicant: Father Flanagan's Boys' Home Doing Business as Boys Town National Research Hospital, Omaha, NE (US)

(72) Inventors: M. Rohan Fernando, Omaha, NE (US); Jianbing Qin, Omaha, NE (US)

(21) Appl. No.: 18/176,152

(22) Filed: Feb. 28, 2023

Related U.S. Application Data
(60) Provisional application No. 63/315,373, filed on Mar. 1, 2022.

(51) Int. Cl. C12Q 1/6883

(52) U.S. Cl. CPC C12Q 1/6883; C12Q 2600/118; C12Q 2600/156; C12Q 2600/16; C12Q 1/6869

(57) ABSTRACT

The present disclosure provides kits, methods, and assays for detecting one or more mutations associated with hereditary or syndromic hearing loss. The method can comprise performing a multigene panel sequencing assay on a biological sample extracted from a subject to identify one or more mutations associated with hearing loss. The disclosed kits, methods, and assays may be particularly useful for determining whether an infant patient is a carrier for or is at risk for developing a hereditary hearing loss-related disorder such as Usher syndrome, Pendred syndrome, Jervell syndrome, and Lange-Nielsen syndrome.

### CROSS REFERENCE TO RELATED APPLICATIONS

This application claims priority under 35 U.S.C. § 119 to provisional patent application U.S. Ser. No. 63/315,373, filed Mar. 1, 2022. The provisional patent application is herein incorporated by reference in its entirety, including without limitation, the specification, claims, and abstract, as well as any figures, tables, appendices, or drawings thereof.

### TECHNICAL FIELD

The present disclosure provides rapid and non-invasive kits and methods for personalized genetic testing of a subject. In some embodiments, a sequencing assay is performed on a biological sample from the subject, which then leads to genetic information related to the subject. The disclosed methods are particularly useful for determining whether an infant patient is at risk for developing a hereditary hearing loss-related disorder such as Usher syndrome, Pendred syndrome, Jervell syndrome, and Lange-Nielsen syndrome.

### BACKGROUND

All newborn babies are screened for hearing problems. If a hearing problem is identified in the newborn, follow-up testing is conducted. Follow-up testing typically includes an assessment of hearing, eyesight, and balance to diagnose hereditary hearing loss disorders such as Usher syndrome. Usher syndrome is an inherited autosomal recessive genetic disease that leads to deafness, visual impairment, and variable degrees of vestibular dysfunction. The subdivisions of Usher Syndrome include type 1, 2, and 3. Since the gene is recessive, the condition only occurs when a patient has two copies of the mutation (homozygous). A carrier of the disease will only have one copy of the mutation (heterozygous).

Once diagnosed, a genetic test may be ordered to determine the type of Usher syndrome or other hearing loss-related disorder. For newborns with genetic disorders, a rapid diagnosis of diseases can make the difference between life and death and reduce length of stay in the neonatal intensive care unit. Genetic testing is the only way to get a definitive diagnosis of some genetic disorders such as Usher syndrome, Pendred syndrome, Jervell syndrome, Lange-Nielsen syndrome, and other hearing loss-associated disorders. Considering the genetic heterogeneity found in mutant genes responsible for hearing impairment, next-generation sequencing (“NGS”) has emerged as a particularly effective tool for the detection of these inherited diseases. However, currently available NGS kits test only a narrow range of mutations for a limited number of hearing loss-related disorders.

Further to the above, NGS is a particularly useful tool to diagnosis genetic disorders because of its ability to detect multiple gene alterations in a single assay in a high throughput fashion. However, the procedures associated with collecting and preparing nucleic acids from biological samples (e.g., blood) are usually cumbersome, and often require specialized equipment or technical skill. Further, these procedures can be time consuming and require a large blood volume that cannot be easily or safely obtained from an infant. In addition, there is a significant financial incentive to shorten newborn patient length of stay and reduce overall patient-management costs associated with delayed or inaccurate diagnosis. Thus, there is a need for rapid and non-invasive methods for determining whether an infant patient is at risk for hereditary hearing loss-related disorders such as Usher syndrome, Pendred syndrome, Jervell syndrome, and Lange-Nielsen syndrome. Given the genetic heterogeneity of hearing impairment, there is also a particular need for a hereditary hearing loss genetic testing assay with a wider array of coverage than current NGS testing kits.

### BRIEF SUMMARY OF PREFERRED EMBODIMENTS

An advantage of this disclosure is that the methods, assays and kits described herein provide the ability to detect at least one mutation of a hereditary hearing loss related gene, including disorders such as Usher syndrome, Pendred syndrome, Jervell syndrome, and Lange-Nielsen syndrome. It is an advantage of the present disclosure within a single assay these disorders can be evaluated and detected.

A preferred embodiment comprises a method for detecting at least one mutation in a plurality of hereditary hearing loss-related genes in a biological sample are provided. In some embodiments, the method comprises extracting genomic DNA from a biological sample obtained from a patient, generating a library comprising a plurality of bait-captured gene sequences corresponding to a plurality of hereditary hearing loss-related genes, wherein the plurality of hereditary hearing loss-related genes comprises three or more of ABHD12, ADGRV1, ARSG, CDH23, CEP250, CEP78, CIB2, CLRN1, ESPN, FOX11, GJB2, GJB6, HARS2, KCNE1, KCNJ10, KCNQ1, MYO7A, PCDH15, PDZD7, SLC26A4, USH1C, USH1G, USH2A, and WHRN; and detecting at least one mutation in at least one of the plurality of bait-captured gene sequences. In other embodiments, the plurality of hereditary hearing loss-related genes comprises ABHD12, ADGRV1, ARSG, CDH23, CEP250, CEP78, CIB2, CLRN1, ESPN, FOX11, GJB2, GJB6, HARS2, KCNE1, KCNJ10, KCNQ1, MYO7A, PCDH15, PDZD7, SLC26A4, USH1C, USH1G, USH2A, and WHRN.

Another preferred embodiment comprises a method for generating a library for the detection of at least one mutation in a plurality of hereditary hearing loss-related genes in a biological sample are also provided. In some embodiments, the library comprises a plurality of bait-captured gene sequences corresponding to the plurality of hereditary hearing loss-related genes, wherein the plurality of hereditary hearing loss-related genes comprises three or more of ABHD12, ADGRV1, ARSG, CDH23, CEP250, CEP78, CIB2, CLRN1, ESPN, FOX11, GJB2, GJB6, HARS2, KCNE1, KCNJ10, KCNQ1, MYO7A, PCDH15, PDZD7, SLC26A4, USH1C, USH1G, USH2A, and WHRN. In some embodiments. the plurality of hereditary hearing loss-related genes comprises ABHD12, ADGRV1, ARSG, CDH23, CEP250, CEP78, CIB2, CLRN1, ESPN, FOX11, GJB2, GJB6, HARS2, KCNE1, KCNJ10, KCNQ1, MYO7A, PCDH15, PDZD7, SLC26A4, USH1C, USH1G, USH2A, and WHRN.

Still a further preferred embodiment comprises a method for detecting at least one mutation in a plurality of hereditary hearing loss-related genes in a biological sample are provided. In some embodiments, the plurality of hereditary hearing loss-related genes comprises three or more of ABHD12, ADGRV1, ARSG, CDH23, CEP250, CEP78, CIB2, CLRN1, ESPN, FOX11, GJB2, GJB6, HARS2, KCNE1, KCNJ10, KCNQ1, MYO7A, PCDH15, PDZD7, SLC26A4, USH1C, USH1G, USH2A, and WHRN. In some embodiments. the plurality of hereditary hearing loss-related genes comprises ABHD12, ADGRV1, ARSG, CDH23, CEP250, CEP78, CIB2, CLRN1, ESPN, FOX11, GJB2, GJB6, HARS2, KCNE1, KCNJ10, KCNQ1, MYO7A, PCDH15, PDZD7, SLC26A4, USH1C, USH1G, USH2A, and WHRN.

Yet another preferred embodiment comprises a kit for detecting at least one mutation in a plurality of hereditary hearing loss-related genes in a biological sample are provided. In some embodiments, the kit comprises a biosampling device and a lysis buffer, wherein the plurality of hereditary hearing loss-related genes comprises three or more of ABHD12, ADGRV1, ARSG, CDH23, CEP250, CEP78, CIB2, CLRN1, ESPN, FOX11, GJB2, GJB6, HARS2, KCNE1, KCNJ10, KCNQ1, MYO7A, PCDH15, PDZD7, SLC26A4, USH1C, USH1G, USH2A, and WHRN.

### BRIEF DESCRIPTION OF THE FIGURES

FIG. 1 depicts mutated genes from patient samples that have been linked to syndromic hearing loss. Mutation consequences may be pathological (path), heterozygous variant not necessarily affected (i.e. no clear disease phenotype) (VOU), or likely pathological. Associated diseases include Usher Syndrome types 1-3 (USH), enlarged vestibular aqueducts (EVA), or non-syndromic deafness. Depending on the patient's genotype and mutation, the patient may be a carrier, normal, or affected.

FIG. 2 provides the genes used in one embodiment of the multigene panel, the location of the genes within the human genome (using assembly build GRCh38.p14), and the associated phenotype.

FIG. 3A depicts an exemplary report of an Usher Syndrome plus gene panel next-generation sequencing (NGS) test for an affected patient homozygous for the CLRN1 pathogenic variant associated with Usher Syndrome type 3A.

FIG. 3B depicts an exemplary report of an Usher Syndrome plus gene panel next-generation sequencing (NGS) test for a carrier patient heterozygous for the CDH23 pathogenic variant associated with Usher Syndrome type 1D/F.

FIG. 3C depicts an exemplary report of an Usher Syndrome plus gene panel next-generation sequencing (NGS) test for a healthy donor (control) with no Usher Syndrome mutations.

The figures described herein form part of the specification and are included to further demonstrate certain preferred embodiments aspects of the disclosure. In some instances, some preferred embodiments can be best understood by referring to the accompanying figures in combination with the detailed description presented herein. The description and accompanying figures may highlight a certain specific example, or a certain aspect of one or more preferred embodiments. However, one skilled in the art will understand that portions of the example or aspect may be used in combination with other examples or aspects of the disclosure.

### DETAILED DESCRIPTION

It is important to identify individuals with hereditary hearing loss efficiently and in a cost-effective manner. Over the last decade, rapid advances in next-generation sequencing (“NGS”) technologies have allowed simultaneous interrogation of multiple genes. NGS technologies perform at higher throughput than Sanger sequencing, since they work in a massively parallel manner. As a result, multigene panel tests utilizing NGS can be a cost-effective and efficient way to detect clinically actionable mutations in appropriately selected patients. Their use may increase detection of pathogenic mutations compared to single-gene testing.

Historically, testing for germline pathogenic or likely pathogenic variants has been performed sequentially through single-gene or single-syndrome testing. However, a multigene panel approach has a number of advantages over the traditional sequential approach. Previous studies have concluded that multigene panel testing compared with single-gene testing can cost-effectively improve the identification of at-risk individuals for early health interventions and the outcome of hearing loss treatment. Studies have also highlighted that panel testing is able to uncover clinically actionable variants unrelated to the syndrome that the clinician initially suspected. Detection of variants is also more common in multigene testing due to the multiplicity of genes tested.

Multigene panel testing thus has emerged as a valuable tool to identify individuals who are at increased risk for hereditary hearing loss. Various targeted NGS-based multigene inherited hearing loss panels have been developed by clinical diagnostic laboratories. As each laboratory-developed test uses different laboratory procedures and bioinformatics pipelines, rigorous laboratory validation is critical to ensure accurate and reliable results from NGS assays for use in clinical practice.

Identification of inherited risk factors via NGS testing allows for health risk mitigation, in addition to increased surveillance and/or surgery. Targeted testing for at-risk infant family members can also subsequently be performed. If positive, providers for the infant family member can take steps to prevent hearing loss or aid in its early detection. Negative results can also reassure the parents of an infant family member and prevent unnecessary surveillance or other preventive measures. Genetic information can also be used to select the patients most appropriate for targeted therapies.

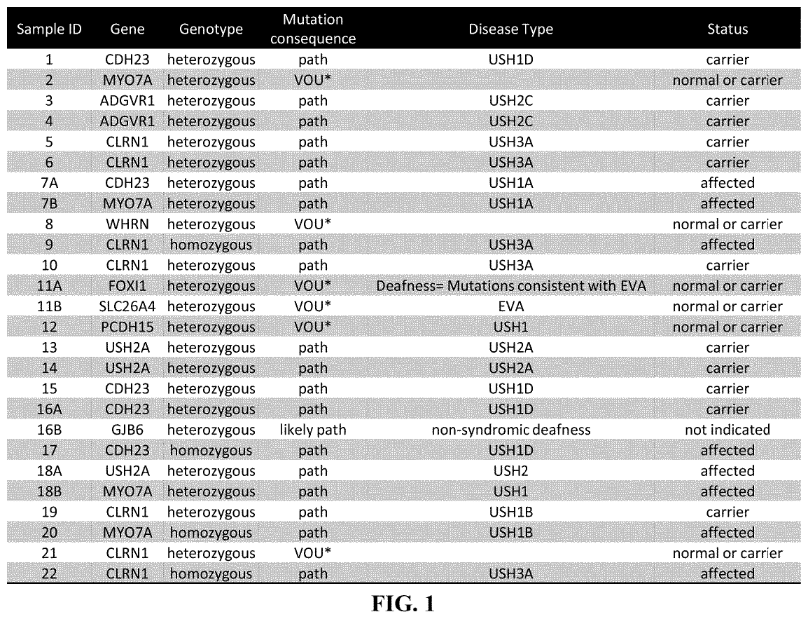

Identifying hereditary hearing loss susceptibility in an infant with a family history can be complex. Pathogenic/likely pathogenic variants leading to the development of hearing loss is often linked to multiple genes. On the other hand, pathogenic/likely pathogenic variants in a single gene can increase the risk for more than one type of hearing loss and/or associated vision loss. FIG. 1 depicts some gene variants identified from patient samples that have been linked to syndromic hearing loss. Outside of identifying actionable variants in genes incidental to the initially suspected syndrome, previous studies have also established that the rate of families carrying more than one actionable variant is higher than what was initially thought when guidelines recommended cascade testing for only the known familial pathogenic/likely pathogenic variants.

Several professional societies have published guidelines that support and define genetic testing for various hereditary syndromes. These societies acknowledge that multigene panel testing may benefit individuals when their histories are consistent with multiple possible hereditary syndromes or when a syndrome can be caused by multiple genes. These panels are also informative when family history information is limited and/or when the history of hearing loss is strong but targeted testing has been negative.

The present disclosure describes the development and validation of a 24-gene inherited hearing loss predisposition panel using NGS for single-nucleotide variants (SNVs), insertions and deletions (Indels), and other variants. Notably, single-nucleotide variants include single-nucleotide polymorphisms (SNPs), and the like. The present disclosure further provides variant detection yield of the panel by summarizing deidentified results from at least three patient specimens for clinical testing with the 24-gene panel.

While the following terms are believed to be well understood by one of ordinary skill in the art, the following definitions are set forth to facilitate explanation of the presently disclosed subject matter.

The term “a” or “an” may refer to one or more of that entity, i.e. can refer to plural referents. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾. This applies regardless of the breadth of the range.

Reference throughout this specification to “one embodiment” or “an embodiment” means that a particular feature, structure, or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics can be combined in any suitable manner in one or more embodiments.

The term “about,” as used herein, refers to variation in the numerical quantity that can occur, for example, through typical measuring techniques and equipment, with respect to any quantifiable variable, including, but not limited to, concentration, mass, time, volume, concentration, temperature, homology, nucleotide count, percentages (including, but not limited to percent hearing loss), etc. As used herein, the terms “about” or “approximately” when preceding a numerical value indicates the value plus or minus a range of 10% of the value. Whether or not modified by the term “about,” the claims include equivalents to the quantities.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

The term “adaptor” refers to a short, chemically synthesized, nucleic acid sequence which can be used to ligate to the end of a nucleic acid sequence to facilitate attachment to another molecule. The adaptor can be single-stranded or double-stranded. An adaptor can incorporate a short (typically less than 50 base pairs) sequence useful for PCR amplification or sequencing.

As used herein, an “alteration” of a nucleic acid sequence, a gene, or a gene product (e.g., a primer, a marker gene, or gene product) refers to the presence of a mutation or mutations within the gene or gene product, e.g., a mutation, which affects the quantity or activity of the gene or gene product, as compared to the normal or wild-type gene. The genetic alteration can result in changes in the quantity, structure, and/or activity of the gene or gene product in a hearing loss tissue or hearing loss cell, as compared to its quantity, structure, and/or activity, in a normal or healthy tissue or cell (e.g., a control). For example, an alteration which is associated with hearing loss can have an altered nucleotide sequence (e.g., a mutation), amino acid sequence, chromosomal translocation, intra-chromosomal inversion, copy number, expression level, protein level, protein activity, in a hearing loss tissue or hearing loss cell, as compared to a normal, healthy tissue or cell.

Exemplary mutations include, but are not limited to, point mutations (e.g., silent, missense, or nonsense), deletions, insertions, inversions, linking mutations, duplications, translocations, inter- and intra-chromosomal rearrangements. Mutations can be present in the coding or non-coding region of the gene. In certain embodiments, the alterations are associated with a phenotype, e.g., a hearing loss phenotype (e.g., one or more of hearing loss risk, hearing loss progression, hearing loss treatment or resistance to hearing loss treatment).

As used herein, an “amount” of an analyte in a body fluid sample refers generally to an absolute value reflecting the mass of the analyte detectable in volume of sample. However, an amount also contemplates a relative amount in comparison to another analyte amount. For example, an amount of an analyte in a sample can be an amount which is greater than a control or normal level of the analyte normally present in the sample.

As used herein, the terms “amplify” or “amplification” with respect to nucleic acid sequences, refer to methods that increase the representation of a population of nucleic acid sequences in a sample. Copies of a particular target nucleic acid sequence generated in vitro in an amplification reaction are called “amplicons” or “amplification products”. Amplification may be exponential or linear. A target nucleic acid may be DNA (such as, for example, genomic DNA and cDNA) or RNA. While the exemplary methods described hereinafter relate to amplification using polymerase chain reaction (PCR), numerous other methods such as isothermal methods, rolling circle methods, etc., are well known to the skilled artisan. The skilled artisan will understand that these other methods may be used either in place of, or together with, PCR methods. See, e.g., Saiki, “Amplification of Genomic DNA” in PCR PROTOCOLS, Innis et ah, Eds., Academic Press, San Diego, Calif. 1990, pp 13-20; Wharam, et al., Nucleic Acids Res. 29(11):E54-E54 (2001).

As used herein, the term “bait” is a type of hybrid capture reagent that retrieves target nucleic acid sequences for sequencing. A bait can be a nucleic acid molecule or a nucleic acid molecule probe, e.g., a DNA or RNA molecule, which can hybridize to (e.g., be complementary to), and thereby allow capture of a target nucleic acid. In one embodiment, a bait is an RNA molecule (e.g., a naturally-occurring or modified RNA molecule); a DNA molecule (e.g., a naturally-occurring or modified DNA molecule), or a combination thereof. In other embodiments, a bait includes a binding entity, e.g., an affinity tag, that allows capture and separation, e.g., by binding to a binding entity, of a hybrid formed by a bait and a target nucleic acid hybridized to the bait. In one embodiment, a bait is suitable for solution phase hybridization. As used herein, “bait set” refers to one or a plurality of bait molecules.

As used herein, the terms “hearing loss” or “deafness” are used interchangeably and refer to the condition of lacking the power of hearing or having impaired hearing. A patient who is not able to hear as well as someone with normal hearing—hearing thresholds of 20 dB or better in both ears—is said to have “hearing loss”. Hearing loss may be mild, moderate, severe, or profound. It can affect one ear or both ears, and leads to difficulty in hearing conversational speech or loud sounds. A “deaf” patient is one who has profound hearing loss, which implies very little or no hearing.

As used herein, the terms “complement”, “complementary” or “complementarity” with reference to polynucleotides (i.e., a sequence of nucleotides such as an oligonucleotide or a target nucleic acid) refer to the Watson/Crick base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, the sequence “5′-A-G-T-3′” is complementary to the sequence “3′-T-C-A-5′”. Certain bases not commonly found in naturally-occurring nucleic acids may be included in the nucleic acids described herein. These include, for example, inosine, 7-deazaguanine, Locked Nucleic Acids (LNA), and Peptide Nucleic Acids (PNA).

Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complement sequence can also be an RNA sequence complementary to the DNA sequence or its complement sequence, and can also be a cDNA.

As used herein, the term “substantially complementary” means that two sequences hybridize under stringent hybridization conditions. The skilled artisan will understand that substantially complementary sequences need not hybridize along their entire length. In particular, substantially complementary sequences may comprise a contiguous sequence of bases that do not hybridize to a target sequence, positioned 3′ or 5′ to a contiguous sequence of bases that hybridize under stringent hybridization conditions to a target sequence.

As used herein, a “control” is an alternative sample used in an experiment for comparison purposes. A control can be “positive” or “negative.” A “control nucleic acid sample” or “reference nucleic acid sample” as used herein, refers to nucleic acid molecules from a control or reference sample. In certain embodiments, the reference or control nucleic acid sample is a wild type or a non-mutated DNA or RNA sequence. In certain embodiments, the reference nucleic acid sample is purified or isolated (e.g., it is removed from its natural state). In other embodiments, the reference nucleic acid sample is from a non-tumor sample, e.g., a blood control, a normal adjacent tumor (NAT), or any other non-hearing loss sample from the same or a different subject.

As used herein, the term “detecting” refers to determining the presence of a mutation or alteration in a nucleic acid of interest in a sample. Detection does not require the method to provide 100% sensitivity.

As used herein, the term “effective amount” refers to a quantity sufficient to achieve a desired therapeutic and/or prophylactic effect, e.g., an amount which results in the prevention of, or a decrease in hereditary hearing loss, or one or more symptoms associated with hereditary hearing loss.

As used herein, the terms “extraction” or “isolation” refer to any action taken to separate nucleic acids from other cellular material present in the sample. The term extraction or isolation includes mechanical or chemical lysis, addition of detergent or protease, or precipitation and removal of other cellular material.

As used herein, the term “gene” refers to a DNA sequence that comprises regulatory and coding sequences necessary for the production of an RNA, which may have a non-coding function (e.g., a ribosomal or transfer RNA) or which may include a polypeptide or a polypeptide precursor. The RNA or polypeptide may be encoded by a full-length coding sequence or by any portion of the coding sequence so long as the desired activity or function is retained. Although a sequence of the nucleic acids may be shown in the form of DNA, a person of ordinary skill in the art recognizes that the corresponding RNA sequence will have a similar sequence with the thymine being replaced by uracil, i.e., “T” is replaced with “U.”

As used herein, the term “genotype” refers to the genetic makeup of an individual cell, cell culture, tissue, organism (e.g., a human), or group of organisms.

As used herein, the term “homologous” or “homologue” or “ortholog” is known in the art and refers to related sequences that share a common ancestor or family member and are determined based on the degree of sequence identity. The terms “homology,” “homologous,” “substantially similar” and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant disclosure such as deletion or insertion of one or more nucleotides that do not substantially alter the functional properties of the resulting nucleic acid fragment relative to the initial, unmodified fragment. It is therefore understood, as those skilled in the art will appreciate, that the disclosure encompasses more than the specific exemplary sequences. These terms describe the relationship between a gene found in one individual, species, or subspecies, and the corresponding or equivalent gene in another individual, species, or subspecies. For purposes of this disclosure, homologous sequences are compared. “Homologous sequences” or “homologues” or “orthologs” are thought or known to be functionally related. A functional relationship may be indicated in any number of ways, including, but not limited to: (a) degree of sequence identity and/or (b) the same or similar biological function. Preferably, both (a) and (b) are indicated. Homology can be determined using software programs readily available in the art, such as those discussed in Current Protocols in Molecular Biology (F. M. Ausubel et ak, eds., 1987) Supplement 30, section 7.718, Table 7.71. Some alignment programs are MacVector (Oxford Molecular Ltd, Oxford, U.K.), ALIGN Plus (Scientific and Educational Software, Pennsylvania) and AlignX (Vector NTI, Invitrogen, Carlsbad, Calif.). Another alignment program is Sequencher (Gene Codes, Ann Arbor, Mich.), using default parameters.

As used herein, the term “hybridize” refers to a process where two substantially complementary nucleic acid strands (at least about 65% complementary over a stretch of at least 14 to 25 nucleotides, at least about 75%, or at least about 90% complementary) anneal to each other under appropriately stringent conditions to form a duplex or heteroduplex through formation of hydrogen bonds between complementary base pairs. Hybridizations are typically and preferably conducted with probe-length nucleic acid molecules, preferably 15-100 nucleotides in length, more preferably 18-50 nucleotides in length. Nucleic acid hybridization techniques are well known in the art. See, e.g., Sambrook, et al., 1989, Molecular Cloning: A Laboratory Manual, Second Edition, Cold Spring Harbor Press, Plainview, N.Y. Hybridization and the strength of hybridization (i.e., the strength of the association between the nucleic acids) is influenced by such factors as the degree of complementarity between the nucleic acids, stringency of the conditions involved, and the thermal melting point (Tₘ) of the formed hybrid. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology, John Wiley & Sons, Secaucus, N.J. In some embodiments, specific hybridization occurs under stringent hybridization conditions. An oligonucleotide or polynucleotide (e.g., a probe or a primer) that is specific for a target nucleic acid will “hybridize” to the target nucleic acid under suitable conditions.

As used herein, the terms “individual”, “patient”, or “subject” can be an individual organism, a vertebrate, a mammal, or a human. In a preferred embodiment, the individual, patient or subject is a human.

As used herein, the term “library” refers to a collection of nucleic acid sequences, e.g., a collection of nucleic acids derived from whole genomic, subgenomic fragments, cDNA, cDNA fragments, RNA, RNA fragments, or a combination thereof. In one embodiment, a portion or all of the library nucleic acid sequences comprises an adaptor sequence. The adaptor sequence can be located at one or both ends. The adaptor sequence can be useful, e.g., for a sequencing method (e.g., an NGS method), for amplification, for reverse transcription, or for cloning into a vector.

In some embodiments, the library comprises a collection of nucleic acid sequences, e.g., a target nucleic acid sequence (e.g., a tumor nucleic acid sequence), a reference nucleic acid sequence, or a combination thereof). In some embodiments, the nucleic acid sequences of the library can be derived from a single subject. In other embodiments, a library can comprise nucleic acid sequences from more than one subject (e.g., 2, 3, 4, 5, 6, 7, 8, 9, 10, 20, 30 or more subjects). In some embodiments, two or more libraries from different subjects can be combined to form a library having nucleic acid sequences from more than one subject. In one embodiment, the subject is an infant having, or at risk of having, hereditary hearing loss.

As used herein, a “library nucleic acid sequence” refers to a nucleic acid molecule, e.g., a DNA, RNA, or a combination thereof, that is a member of a library. Typically, a library nucleic acid sequence is a DNA molecule, e.g., genomic DNA or cDNA. In some embodiments, a library nucleic acid sequence is fragmented, e.g., sheared or enzymatically prepared, genomic DNA. In certain embodiments, the library nucleic acid sequences comprise sequence from a subject and sequence not derived from the subject, e.g., adaptor sequence, a primer sequence, or other sequences that allow for identification, e.g., “barcode” sequences.

As used herein, “next generation sequencing” or “NGS” refers to any sequencing method that determines the nucleotide sequence of either individual nucleic acid molecules (e.g., in single molecule sequencing) or clonally expanded proxies for individual nucleic acid molecules in a high throughput parallel fashion (e.g., greater than 10³, 10⁴, 10⁵ or more molecules are sequenced simultaneously). In one embodiment, the relative abundance of the nucleic acid species in the library can be estimated by counting the relative number of occurrences of their cognate sequences in the data generated by the sequencing experiment. Next generation sequencing methods are known in the art, and are described, e.g., in Metzker, M. Nature Biotechnology Reviews 11 :31-46 (2010).

As used herein, the term “nucleic acid” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like. The terms “nucleic acid” and “nucleotide sequence” are used interchangeably.

As used herein, “oligonucleotide” refers to a molecule that has a sequence of nucleic acid bases on a backbone comprised mainly of identical monomer units at defined intervals. The bases are arranged on the backbone in such a way that they can bind with a nucleic acid having a sequence of bases that are complementary to the bases of the oligonucleotide. The most common oligonucleotides have a backbone of sugar phosphate units. A distinction may be made between oligodeoxyribonucleotides that do not have a hydroxyl group at the 2′ position and oligoribonucleotides that have a hydroxyl group at the 2′ position. Oligonucleotides may also include derivatives, in which the hydrogen of the hydroxyl group is replaced with organic groups, e.g., an allyl group. Oligonucleotides that function as primers or probes are generally at least about 10-15 nucleotides in length or up to about 70, 100, 110, 150 or 200 nucleotides in length, and more preferably at least about 15 to 25 nucleotides in length, although shorter or longer oligonucleotides may be used in the method. The exact size will depend on many factors, which in turn depend on the ultimate function or use of the oligonucleotide. The oligonucleotide may be generated in any manner, including, for example, chemical synthesis, DNA replication, restriction endonuclease digestion of plasmids or phage DNA, reverse transcription, PCR, or a combination thereof. The oligonucleotide may be modified e.g., by addition of a methyl group, a biotin or digoxigenin moiety, a fluorescent tag or by using radioactive nucleotides.

Oligonucleotides used as primers or probes for specifically amplifying or specifically detecting a particular target nucleic acid generally are capable of specifically hybridizing to the target nucleic acid.

As used herein, the term “phenotype” refers to the observable characteristics of an individual cell, cell culture, organism (e.g., a human), or group of organisms which results from the interaction between that individual's genetic makeup (i.e., genotype) and the environment.

As used herein, the term “primer” refers to an oligonucleotide, which is capable of acting as a point of initiation of nucleic acid sequence synthesis when placed under conditions in which synthesis of a primer extension product which is complementary to a target nucleic acid strand is induced, i.e., in the presence of different nucleotide triphosphates and a polymerase in an appropriate buffer (“buffer” includes pH, ionic strength, cofactors etc.) and at a suitable temperature. One or more of the nucleotides of the primer can be modified for instance by addition of a methyl group, a biotin or digoxigenin moiety, a fluorescent tag or by using radioactive nucleotides. A primer sequence need not reflect the exact sequence of the template. For example, a non-complementary nucleotide fragment may be attached to the 5′ end of the primer, with the remainder of the primer sequence being substantially complementary to the strand. The term primer as used herein includes all forms of primers that may be synthesized including peptide nucleic acid primers, locked nucleic acid primers, phosphorothioate modified primers, labeled primers, and the like. The term “forward primer” as used herein means a primer that anneals to the anti-sense strand of double-stranded DNA (dsDNA). A “reverse primer” anneals to the sense-strand of dsDNA.

In some embodiments, primers are at least about 10, 15, 18, or 30 nucleotides in length or up to about 100, 110, 125, or 200 nucleotides in length. In some embodiments, primers are preferably between about 15 to about 60 nucleotides in length, and most preferably between about 25 to about 40 nucleotides in length. In some embodiments, primers are about 15 to about 35 nucleotides in length. There is no standard length for optimal hybridization or polymerase chain reaction amplification. An optimal length for a particular primer application may be readily determined in the manner described in H. Erlich, PCR Technology, PRINCIPLES AND APPLICATION FOR DNA AMPLIFICATION, (1989).

As used herein, the term “primer pair” refers to a forward and reverse primer pair (i.e., a left and right primer pair) that can be used together to amplify a given region of a nucleic acid of interest.

As used herein, the term “probe” refers to a nucleic acid sequences that interacts with a target nucleic acids via hybridization. A probe may be fully complementary to a target nucleic acid sequence or partially complementary. The level of complementarity will depend on many factors based, in general, on the function of the probe. Probes can be labeled or unlabeled, or modified in any of a number of ways well known in the art. A probe may specifically hybridize to a target nucleic acid. Probes may be DNA, RNA or a RNA/DNA hybrid. Probes may be oligonucleotides, artificial chromosomes, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes may comprise modified nucleobases, modified sugar moieties, and modified internucleotide linkages. Probes are typically at least about 10, 15, 20, 25, 30, 35, 40, 50, 60, 75, 100 nucleotides or more in length.

As used herein, the term “sample” refers to clinical samples obtained from a patient. In preferred embodiments, a sample is obtained from a biological source (i.e., a “biological sample”), such as tissue or bodily fluid collected from a subject. Sample sources include, but are not limited to, mucus, sputum (processed or unprocessed), bronchial alveolar lavage (BAL), bronchial wash (BW), blood, bodily fluids, cerebrospinal fluid (CSF), urine, plasma, serum, or tissue (e.g., biopsy material). Preferred sample sources include plasma, serum, or whole blood.

As used herein, the term “sensitivity,” in reference to the methods of the present technology, is a measure of the ability of a method to detect a preselected sequence variant in a heterogeneous population of sequences. A method has a sensitivity of S % for variants of F if, given a sample in which the preselected sequence variant is present as at least F % of the sequences in the sample, the method can detect the preselected sequence at a preselected confidence of C %, S % of the time. By way of example, a method has a sensitivity of 90% for variants of 5% if, given a sample in which the preselected variant sequence is present as at least 5% of the sequences in the sample, the method can detect the preselected sequence at a preselected confidence of 99%, 9 out of 10 times (F=5%; C=99%; S=90%). Exemplary sensitivities include at least 50, 60, 70, 80, 90, 95, 98, and 99%.

As used herein, the term “specific” in reference to an oligonucleotide primer means that the nucleotide sequence of the primer has at least 12 bases of sequence identity with a portion of the nucleic acid to be amplified when the oligonucleotide and the nucleic acid are aligned. An oligonucleotide primer that is specific for a nucleic acid is one that, under the stringent hybridization or washing conditions, is capable of hybridizing to the target of interest and not substantially hybridizing to nucleic acids which are not of interest. Higher levels of sequence identity are preferred and include at least 75%, at least 80%, at least 85%, at least 90%, at least 85-95% and more preferably at least 98% sequence identity. Sequence identity can be determined using a commercially available computer program with a default setting that employs algorithms well known in the art. As used herein, sequences that have “high sequence identity” have identical nucleotides at least at about 50% of aligned nucleotide positions, preferably at least at about 60% of aligned nucleotide positions, and more preferably at least at about 75% of aligned nucleotide positions.

As used herein, the term “specificity” is a measure of the ability of a method to distinguish a truly occurring preselected sequence variant from sequencing artifacts or other closely related sequences. It is the ability to avoid false positive detections. False positive detections can arise from errors introduced into the sequence of interest during sample preparation, sequencing error, or inadvertent sequencing of closely related sequences like pseudo-genes or members of a gene family. A method has a specificity of X % if, when applied to a sample set of N-Total sequences, in which X-me sequences are truly variant and X-Nottme are not truly variant, the method selects at least X % of the not truly variant as not variant. E.g., a method has a specificity of 90% if, when applied to a sample set of 1,000 sequences, in which 500 sequences are truly variant and 500 are not truly variant, the method selects 90% of the 500 not truly variant sequences as not variant. Exemplary specificities include at least 50, 60, 70, 80, 90, 95, 98, and 99%.

As used herein, the terms “target nucleic acid” or “target sequence” as used herein refer to a nucleic acid sequence of interest to be detected and/or quantified in the sample to be analyzed. Target nucleic acid may be composed of segments of a chromosome, a complete gene with or without intergenic sequence, segments or portions of a gene with or without intergenic sequence, or sequence of nucleic acids which probes or primers are designed. Target nucleic acids may include a wild-type sequence(s), a mutation, deletion, insertion or duplication, tandem repeat elements, a gene of interest, a region of a gene of interest or any upstream or downstream region thereof. Target nucleic acids may represent alternative sequences or alleles of a particular gene. Target nucleic acids may be derived from genomic DNA, cDNA, or RNA.

As used herein, the terms “treat,” “treating” or “treatment” refer, to an action to obtain a beneficial or desired clinical result including, but not limited to, alleviation or amelioration of one or more signs or symptoms of a disease or condition, diminishing the extent of disease, stability (i.e., not worsening, achieving stable disease) state of disease, amelioration or palliation of the disease state, and diminishing rate of or time to progression.

As used herein, the phrase “Usher syndrome” refers to an inherited autosomal recessive genetic disease that leads to deafness, visual impairment, and variable degrees of vestibular dysfunction. Since the gene is recessive, the condition only occurs when a patient has two copies of the mutation (homozygous). A carrier of the disease will only have one copy of the mutation (heterozygous). Usher syndrome is characterized by sensorineural hearing loss, retinitis pigmentosa (RP) and in some cases, vestibular dysfunction. Usher syndrome is a clinically and genetically heterogeneous disease, accounting for about half of all cases of combined hereditary deafness-blindness. Three clinical forms of the disease have been identified (USH I, II, and III) based on the severity of the hearing impairment, the presence or absence of vestibular dysfunction, and the age of onset of the disease. Usher syndrome Type II is the most frequent clinical form accounting for approximately 50% of all Usher syndrome cases. Usher syndrome Type II is characterized by congenital hearing loss and progressive vision loss starting in adolescence or adulthood. The hearing loss ranges from mild to severe and mainly affects the ability to hear high-frequency sounds. Vision loss occurs as the light-sensing cells of the retina gradually deteriorate. Night vision loss begins first, followed by loss of the peripheral vision. With time, these blind areas enlarge and merge to produce tunnel vision. In some cases, vision is further impaired by cataracts. Many patients become legally blind in the 5^{th} decade of life. Usher syndrome Type 2A is due to a mutation in the USH2A gene and accounts for approximately 80% of all Usher syndrome Type II cases and 40% of all Usher syndrome cases.

The present technology is not to be limited in terms of the particular embodiments described in this application, which are intended as single illustrations of individual embodiments of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting

In some embodiments, the disclosure is drawn a multigene inherited hearing loss predisposition panel utilized for identifying heredity hearing loss susceptibility in an individual. In some embodiments, multigene refers to, within the context of the panel, a screen for at least two genes of interest.

In some embodiments, multigene refers to, within the context of the panel, a screen for at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, or at least 35 genes of interest.

In some embodiments, multigene refers to, within the context of the panel, a screen for at least 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, or at least about 35 genes of interest.

In some embodiments, the multigene inherited hearing loss predisposition panel utilizes at least two genes selected from the following: MYO7A, USH1C, CDH23, PCDH15, USH1G, CIB2, USH2A, ADGRV1, WHRN, CLRN1, HARS2, SLC26A4, FOXI1, KCNJ10, KCNE1, KCNQ1, ABHD12, PDZD7, ESPN, ARSG, CEP250, CEP78, GJB2, and GJB6. In some embodiments, the multigene inherited hearing loss predisposition panel utilizes the following 24 genes: MYO7A, USH1C, CDH23, PCDH15, USH1G, CIB2, USH2A, ADGRV1, WHRN, CLRN1, HARS2, SLC26A4, FOXI1, KCNJ10, KCNE1, KCNQ1, ABHD12, PDZD7, ESPN, ARSG, CEP250, CEP78, GJB2, and GJB6. In other embodiments, the multigene inherited hearing loss predisposition panel also utilizes the following genes: USH1B, USH1D, USH1E, USH1F, USH1G, USH2C, USH2D, USH3A, JLNS1, JLNS2, COL4A3, HARS1, COL4A4, COL4A5, EYA1, SIX5, SIX1, SEMA3E, CHD7, NDP, HSD17B4, CLPP, LARS2, TWNK, ERAL1, COL2A1, COL11A1, COL11A2, COL9A1, COL9A2, TCOF1, POLR1D, POLR1C, SANS, VLGR1/GPR98, PAX3, MITF, SNAI2, SOX10, PAX3, EDNRB, EDN3, and/or SOX10.

In some embodiments, the multigene inherited hearing loss predisposition panel utilizes at least two genes represented by the example transcript IDs provided in Table 1 or Table 2. In some embodiments, the multigene inherited hearing loss predisposition panel utilizes the 20 genes represented by the example transcript IDs provided in Table 1 or the 24 genes represented by the example transcript IDs provided in Table 2. In other embodiments, a multigene inherited hearing loss predisposition panel using up to 75 genes is contemplated.

In some embodiments, the multigene inherited hearing loss predisposition panel is capable of detecting one or more variants of the genes provided in Table 1. In some embodiments, detecting at least one mutation in at least one of the above-described plurality of bait-captured gene sequences indicates an increased susceptibility to hereditary hearing loss in a patient. Notably, as described above, the multigene inherited hearing loss predisposition panel is also referred to herein as “Usher syndrome Plus NGS testing assay”, “Usher syndrome Plus NGS multigene panel assay”, and/or “NGS multigene hearing loss predisposition panel”.

In some embodiments, the disclosure is generally drawn to a method for detecting at least one mutation in a plurality of hereditary hearing loss-related genes in a biological sample, the method comprising: (a) extracting genomic DNA from a biological sample obtained from a patient, (b) generating a library comprising a plurality of bait-captured gene sequences corresponding to a plurality of hereditary hearing loss-related genes, wherein the plurality of hereditary hearing loss-related genes comprises three or more of ABHD12, ADGRV1, ARSG, CDH23, CEP250, CEP78, CIB2, CLRN1, ESPN, FOX11, GJB2, GJB6, HARS2, KCNE1, KCNJ10, KCNQ1, MYO7A, PCDH15, PDZD7, SLC26A4, USH1C, USH1G, USH2A, and WHRN; and (c) detecting at least one mutation in at least one of the plurality of bait-captured gene sequences.

In some embodiments, the plurality of hearing loss-related genes comprises one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, or all twenty-four (24) of ABHD12, ADGRV1, ARSG, CDH23, CEP250, CEP78, CIB2, CLRN1, ESPN, FOX11, GJB2, GJB6, HARS2, KCNE1, KCNJ10, KCNQ1, MYO7A, PCDH15, PDZD7, SLC26A4, USH1C, USH1G, USH2A, and WHRN. The plurality of hearing loss-related genes may also include other gene variants or mutations associated with Usher syndrome, Pendred syndrome, Jervell syndrome, Lange-Nielsen syndrome, or other syndromic or nonsyndromic causes of hearing loss, including variants not yet identified. Thus, this disclosure is not limited to the 24 genes listed in Table 2.

In some embodiments, the method comprises: (a) extracting genomic DNA from a biological sample obtained from a patient, (b) generating a library comprising a plurality of bait-captured gene sequences corresponding to each of the plurality of hereditary hearing loss-related genes from the genomic DNA extracted in (a), the plurality of hereditary hearing loss-related genes comprising MYO7A, USH1C, CDH23, PCDH15, USH1G, CIB2, USH2A, ADGRV1, WHRN, CLRN1, HARS2, SLC26A4, FOXI1, KCNJ10, KCNE1, KCNQ1, ABHD12, PDZD7, ESPN, ARSG, CEP250, CEP78, GJB2, and GJB6; and (c) detecting at least one mutation in at least one of the plurality of bait-captured gene sequences using high throughput massive parallel sequencing (e.g., massive parallel sequencing via Illumina NextSeq 550Dx, and the like).

In some embodiments, this next-generation sequencing (“NGS”) assay is designed to detect causative genetic alterations involved in all three types of Usher syndrome, in addition to other hearing loss-related disorders. For example, the NGS multigene hearing loss predisposition panel can detect three other common autosomal recessive syndromic hearing loss disorders (Pendred syndrome, Jervell syndrome, and Lange-Nielsen syndrome). As indicated above, the most common pathogenic variants in genes GJB2 and GJB6 of nonsyndromic hearing loss can also be detected by the panel. Notably, as described above, the multigene inherited hearing loss predisposition panel may also utilize the following genes: USH1B, USH1D, USH1E, USH1F, USH1G, USH2C, USH2D, USH3A, JLNS1, JLNS2, COL4A3, HARS1, COL4A4, COL4A5, EYA1, SIX5, SIX1, SEMA3E, CHD7, NDP, HSD17B4, CLPP, LARS2, TWNK, ERAL1, COL2A1, COL11A1, COL11A2, COL9A1, COL9A2, TCOF1, POLR1D, POLR1C, SANS, VLGR1/GPR98, PAX3, MITF, SNAI2, SOX10, PAX3, EDNRB, EDN3, and/or SOX10.

The herein described Usher Syndrome Plus NGS Testing Assay provides an unexpectedly wide range of coverage for Usher syndrome and related hearing loss-related disorders as compared to alternate available NGS testing kits. This unexpected breadth of coverage is due, in part, to the genetic heterogeneity of hearing impairment and the recent discovery additional mutants falling into the range of coverage of the herein described Usher Syndrome Plus NGS Testing Assay. Demonstrating the breadth of the assay, when compared to currently available Fulgent and GeneDx Usher syndrome panels, at least 8 of the genes shown in Table 1 are not detectable by either commercial panel. Specifically, genes HARS SLC2644, FOXL1, KCNJ10, KCNE1, KCNQ1, ESPN, ARSG, CEP250, CEP78, GJB2, and GJB6 are not detected by the Fulgent and Genex panels.

In some embodiments, the biological sample is plasma, serum, or whole blood. In other embodiments, the biological sample is dried plasma, dried serum, and/or dried whole blood. In one embodiment, the biological sample is a dried human biological sample. In another embodiment, the biological sample is obtained from an infant patient having or suspected of having a hereditary hearing loss disorder (e.g., Usher syndrome, Pendred syndrome, Jervell syndrome, or Lange-Nielsen syndrome). In some embodiments, detecting at least one mutation in at least one of the plurality of bait-captured gene sequences indicates an increased susceptibility to hereditary hearing loss in a patient (e.g., a newborn patient). In certain embodiments, the increased susceptibility to hereditary hearing loss is an increase of at least about 0.5%, 1%, 5%, 10%, 15%, 20%, 25%, 30%, 35%, 40%, 45%, 50%, 55%, 60%, 65%, 70%, 75%, 80%, 85%, 90%, or 95%.

Additionally, in some embodiments, one or both ends of the plurality of bait-captured gene sequences comprise an adaptor sequence. In other embodiments, a subset of the plurality of bait-captured gene sequences comprise an adaptor sequence at one or both ends. Examples of adaptor sequences include P5 adaptors, P7 adaptors, PI adaptors, A adaptors, or Ion Xpress™ barcode adaptors. In some embodiments, Illumina adaptors include P5 and P7 adaptors that allow a library (e.g., a library comprising a plurality of bait-captured gene sequences) to bind and generate clusters on the flow cell. In other embodiments, further adaptors contain sequencing primer binding sites to initiate sequencing (e.g., adaptors Rd1 SP and Rd2 SP). In other examples, adaptors contain index sequences comprising sample identifiers that allow multiplexing/pooling of multiple samples in a single sequencing run or flow cell lane.

In some embodiments, the library is prepared using Agilent's SureSelect XT HS2 kit and sequenced on an Illumina platform (e.g. NextSeq 550Dx). In some embodiments, high throughput massive parallel sequencing is performed using pyrosequencing, reversible dye-terminator sequencing, SOLiD sequencing, Ion semiconductor sequencing, Helioscope single molecule sequencing, sequencing by synthesis, sequencing by ligation, or SMRT™ sequencing.

In some embodiments, the method comprises the detection of genes chosen according to the prevalence of mutations in a given gene associated with hereditary hearing loss. In some embodiments, the multigene inherited hearing loss predisposition panel uses at least 5,000 probes. In some embodiments, at least one of the at least 5,000 nucleic acid probes comprises a region of complementarity to at least one of the plurality of hereditary hearing loss-related genes, the region of complementarity comprising a coding region in addition to 10 bases of an untranslated region (UTR) of the at least one of the plurality of hereditary hearing loss-related genes. In a preferred embodiment, the multigene inherited hearing loss predisposition panel uses at least 5437 probes. In other embodiments, the multigene inherited hearing loss predisposition panel uses at least 6,000 7,000, 8,000, 9,000, or 10,000 probes.

In some embodiments, the method further comprises amplifying GJB2 and GJB6, or any other gene listed in Table 1, using long-range PCR. As is well known in the art, long-range PCR refers to the amplification of DNA lengths that cannot typically be amplified using routine PCR methods or reagents. In some embodiments, polymerases optimized for long-range PCR can amplify up to 30 kb and beyond. In some embodiments, long-range PCR products are subjected to mechanical shearing, enzymatic end repair, and 3′ adenylation. In the present disclosure, GJB2 and GJB6 are described as exemplar genes, but long-range PCR may be applied to any of the above-described hereditary hearing loss-related genes.

In one embodiment, one or more exons of GJB2 are amplified using long-range PCR to generate a plurality of GJB2 amplicons and to detect at least one mutation in at least one of the plurality of GJB2 amplicons using high throughput massive parallel sequencing. The plurality of GJB2 amplicons may or may not include an adaptor sequence. In another embodiment, the method further comprises amplifying GJB6 (e.g., amplifying one or more exons of GJB6) using long-range PCR to generate a plurality of GJB6 amplicons and detecting at least one mutation in at least one of the plurality of GJB6 amplicons using high throughput massive parallel sequencing. The plurality of GJB6 amplicons may or may not include an adaptor sequence.

In certain embodiments, one or more exons of GJB2 are amplified using a forward primer that anneals to the anti-sense strand of the double-stranded DNA (dsDNA) template and a reverse primer that anneals to the sense-strand of dsDNA template to generate the GJB2 amplicons. In certain embodiments, one or more exons of GJB6 are amplified using a forward primer that anneals to the anti-sense strand of the dsDNA template and a reverse primer that anneals to the sense-strand of dsDNA template to generate the GJB2 amplicons.

In some embodiments, the specificity of detecting at least one mutation in GJB2, GJB6, or another hereditary hearing loss-related gene is increased relative to a method that does not perform long-range PCR on GJB2, GJB6, or another hearing loss-related gene prior to performing high throughput massive parallel sequencing. In some embodiments, the increased specificity is an increase of at least 0.5%, 1%, 5%, 10%, 15%, 20%, 25%, 30%, 35%, 40%, 45%, 50%, 55%, 60%, 65%, 70%, 75%, 80%, 85%, 90%, or 95% relative to a corresponding control.

In certain embodiments, the detection interference from pseudogenes is decreased relative to a method that does not perform long-range PCR of GJB2, GJB6, or another hereditary hearing loss-related gene prior to performing high throughput massive parallel sequencing. In some embodiments, the decreased detection interference is a decrease of at least 0.5%, 1%, 5%, 10%, 15%, 20%, 25%, 30%, 35%, 40%, 45%, 50%, 55%, 60%, 65%, 70%, 75%, 80%, 85%, 90%, or 95% relative to a corresponding control.

In another aspect, a method for generating a library for the detection of at least one mutation in a plurality of hereditary hearing loss-related genes in a biological sample is provided. In some embodiments, the library comprises a plurality of bait-captured gene sequences corresponding to the plurality of hereditary hearing loss-related genes. In some embodiments, the plurality of hereditary hearing loss-related genes comprises one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, or all twenty-four (24) of ABHD12, ADGRV1, ARSG, CDH23, CEP250, CEP78, CIB2, CLRN1, ESPN, FOX11, GJB2, GJB6, HARS2, KCNE1, KCNJ10, KCNQ1, MYO7A, PCDH15, PDZD7, SLC26A4, USH1C, USH1G, USH2A, and WHRN.

In some embodiments, kits for use in practicing the methods described herein are contemplated. In some embodiments, kits comprise the multigene inherited hearing loss predisposition panel and all solutions, buffers, and vessels sufficient for performing the methods described herein. In some embodiments, the kit comprises a biosampling device and a lysis buffer, wherein the plurality of hereditary hearing loss-related genes comprises three or more of ABHD12, ADGRV1, ARSG, CDH23, CEP250, CEP78, CIB2, CLRN1, ESPN, FOX11, GJB2, GJB6, HARS2, KCNE1, KCNJ10, KCNQ1, MYO7A, PCDH15, PDZD7, SLC26A4, USH1C, USH1G, USH2A, and WHRN. In an embodiment, the plurality of hereditary hearing loss-related genes comprises all 24 of ABHD12, ADGRV1, ARSG, CDH23, CEP250, CEP78, CIB2, CLRN1, ESPN, FOX11, GJB2, GJB6, HARS2, KCNE1, KCNJ10, KCNQ1, MYO7A, PCDH15, PDZD7, SLC26A4, USH1C, USH1G, USH2A, and WHRN. The biosampling device can be any device known in the art used to collect a biological sample from a patient. In some embodiments, the biosampling device is a volumetric absorptive biosampling device, such as a MITRA® tip. In other embodiments, the biosampling device is a cup, slide, syringe, needle, swab, gauze, paper, pipette, vial, vacutainer, tube, or any other device suitable to collect a biological sample.

In one example, a kit for detecting at least one mutation in a plurality of hereditary hearing loss-related genes in a biological sample is contemplated, the kit comprising a skin puncture tool, a volumetric absorptive biosampling device, and a lysis buffer, wherein the plurality of hereditary hearing loss-related genes comprises MYO7A, USH1C, CDH23, PCDH15, USH1G, CIB2, USH2A, ADGRV1, WHRN, CLRN1, HARS2, SLC26A4, FOXI1, KCNJ10, KCNE1, KCNQ1, ABHD12, PDZD7, ESPN, ARSG, CEP250, CEP78, GJB2, and GJB6. In other embodiments, the plurality of hereditary hearing loss-related genes further comprises: USH1B, USH1D, USH1E, USH1F, USH1G, USH2C, USH2D, USH3A, JLNS1, JLNS2, COL4A3, HARS1, COL4A4, COL4A5, EYA1, SIX5, SIX1, SEMA3E, CHD7, NDP, HSD17B4, CLPP, LARS2, TWNK, ERAL1, COL2A1, COL11A1, COL11A2, COL9A1, COL9A2, TCOF1, POLR1D, POLR1C, SANS, VLGR1/GPR98, PAX3, MITF, SNAI2, SOX10, PAX3, EDNRB, EDN3, and/or SOX10.

In some embodiments, the kit comprises one or more primer pairs that hybridize to one or more regions or exons of one or more of the plurality of hereditary hearing loss-related genes. In other embodiments, the kit further comprises one or more bait sequences that hybridize to one or more regions or exons of one or more of the plurality of hereditary hearing loss-related genes. In some embodiments, the lysis buffer comprises guanidine hydrochloride, Tris.Cl, EDTA, Tween 20, and Triton X-100. In other embodiments, the volumetric absorptive biosampling device is a MITRA® tip.

In some embodiments, the kit comprises a biosampling device with an porous tip having a distal end and a proximal end. The width of the distal end of the porous tip is narrow compared to the width of the proximal end. The proximal end is attached to a holder, whereas the distal end is configured to contact a fluid to be absorbed, such as blood. The biosampling device permits biological fluid samples, such as blood, to be easily dried, shipped, and then later analyzed. In certain embodiments, the biological fluid is blood from a fingerstick. In other embodiments, the biological fluid is blood from a continuous blood monitor. Wicking action draws the blood into the porous tip. An optional barrier between the porous tip and the holder prevents blood from passing or wicking to the holder. The porous tip is composed of a material that wicks up substantially the same volume of fluid even when excess fluid is available. The volume of the porous tip affects the volume of fluid absorbed. The size and shape of the porous tip may be varied to adjust the volume of absorbed blood and the rate of absorption. Blood volumes of about 5-20 μL, about 25 μL and even up to about 35 μL may be acceptable. The sampling time may be about 1 second, about 3 seconds, about 5 seconds, or up to about 15 seconds.

In some embodiments, the material used for the porous tip is hydrophilic (e.g., polyester). Alternatively, the material may initially be hydrophobic and is subsequently treated to make it hydrophilic. Hydrophobic matrices may be rendered hydrophilic by a variety of known methods, such as plasma treatment or surfactant treatment of the matrix. In some embodiments, plasma treatment is used to render a hydrophobic material such as polyolefin, e.g., polyethylene, hydrophilic.

All publications and patent applications mentioned in the specification are indicative of the level of those skilled in the art to which this disclosure pertains.

Although the foregoing disclosure has been described in some detail by way of illustration and example for purposes of clarity of understanding, it will be obvious that certain changes and modifications may be practiced within the scope of the appended claims. Thus, many modifications and other embodiments of the disclosure will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims.

The following examples are offered by way of illustration and not by way of limitation.

### EXAMPLES

Embodiments of the present disclosure are further defined in the following non-limiting Examples. It should be understood that these Examples, while indicating certain embodiments of the disclosure, are given by way of illustration only. From the above discussion and these Examples, one skilled in the art can ascertain the essential characteristics of this disclosure, and without departing from the spirit and scope thereof, can make various changes and modifications of the embodiments of the disclosure to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims.

### Example 1: Gene Selection and DNA Specimens

Twenty genes were selected for the inherited hearing loss predisposition panel. These twenty genes are associated with well-characterized hearing loss disorders, along with more recently discovered genes associated with increased hearing loss risk (Table 1). The genes selected increase the risk of hereditary hearing loss including conductive, sensorineural, central, mixed and functional hearing loss types.

TABLE 1
Exemplar genes comprising the 20-gene inherited
hearing loss predisposition panel.

| Gene Name | Example Transcript ID |
|---|---|
| ABHD12 | NM_001042472.3 |
| ADGRV1 | NM_032119.4 |
| CDH23 | NM_022124.6 |
| CIB2 | NM_001271888.2 |
| CLRN1 | NM_001195794.1 |
| FOXI1 | NM_012188.5 |
| GJB2 | NM_004004.6 |
| GJB6 | NM_001110219.3 |
| HARS2 | NM_001278731.2 |
| KCNE1 | NM_000219.6 |
| KCNJ10 | NM_002241.5 |
| KCNQ1 | NM_000218.3 |
| MYo7A | NM_000260.4 |
| PCDH15 | NM_001142763.2 |
| PDZD7 | NM_001195263.2 |
| SLC26A4 | NM_000441.2 |
| USH1C | NM_005709.4 |
| USH1G | NM_001282489.3 |
| USH2A | NM_007123.6 |
| WHRN | NM_015404.4 |

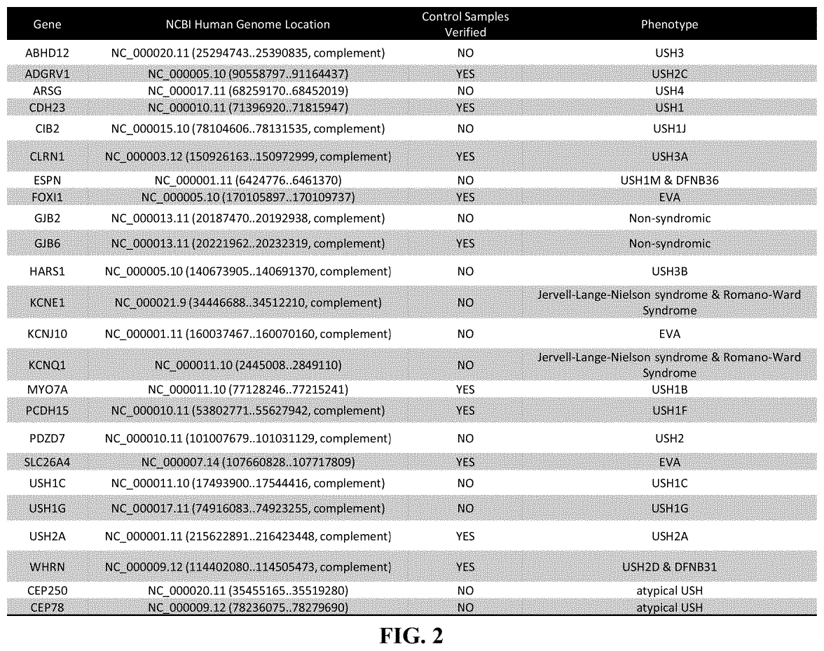

In another embodiment, the multigene inherited hearing loss predisposition panel may include twenty-four genes (Table 2 and FIG. 2).

TABLE 2
Exemplar genes comprising the 24-gene inherited
hearing loss predisposition panel.

| Gene Name | Example Transcript ID |
|---|---|
| ABHD12 | NM_001042472.3 |
| ADGRV1 | NM_032119.4 |
| ARSG | NM_001267727.2 |
| CDH23 | NM_022124.6 |
| CEP78 | NM_001330691.3 |
| CEP250 | NM_007186.6 |
| CIB2 | NM_001271888.2 |
| CLRN1 | NM_001195794.1 |
| ESPN | NM_031475.3 |
| FOXI1 | NM_012188.5 |
| GJB2 | NM_004004.6 |
| GJB6 | NM_001110219.3 |
| HARS2 | NM_001278731.2 |
| KCNE1 | NM_000219.6 |
| KCNJ10 | NM_002241.5 |
| KCNQ1 | NM_000218.3 |
| MY07A | NM_000260.4 |
| PCDH15 | NM_001142763.2 |
| PDZD7 | NM_001195263.2 |
| SLC26A4 | NM_000441.2 |
| USH1C | NM_005709.4 |
| USH1G | NM_001282489.3 |
| USH2A | NM_007123.6 |
| WHRN | NM_015404.4 |

In addition to the exemplar genes shown above, the multigene inherited hearing loss predisposition panel may include other hereditary hearing loss genes known in the art, including but not limited to: USH1B, USH1D, USH1E, USH1F, USH1G, USH2C, USH2D, USH3A, JLNS1, JLNS2, COL4A3, HARS1, COL4A4, COL4A5, EYA1, SIX5, SIX1, SEMA3E, CHD7, NDP, HSD17B4, CLPP, LARS2, TWNK, ERAL1, COL2A1, COL11A1, COL11A2, COL9A1, COL9A2, TCOF1, POLR1D, POLR1C, SANS, VLGR1/GPR98, PAX3, MITF, SNAI2, SOX10, PAX3, EDNRB, EDN3, and/or SOX10. Further, the above example transcript IDs refer to exemplar mRNA/cDNA for each respective gene but are not to be read as limiting. The present disclosure contemplates a variety of potential transcripts for each of the plurality of hereditary hearing loss-related genes disclosed. For example, the transcript for MYO7A is not limited to NM_000260.4, but rather may include other REFSEQ mRNAs such as NM_001127180.2, NM_001369365.1, and NM_001127179.2.

For validation of the 20-gene hereditary hearing loss predisposition panel (“Usher Syndrome Plus NGS Testing Assay”), three de-identified residual whole blood patient specimens were used, representing a patient with Usher Syndrome, a known carrier (e.g., a carrier of one mutant allele), and a healthy donor sample. These specimens were validated for analytical accuracy, sensitivity and specificity using standard protocols. For each patient, informed consent for genetic analysis was obtained. Patient results were de-identified before analysis. As shown below, the testing results confirmed the previous diagnoses of the known Usher syndrome patient and carrier, with no mutations detected in the sample of the healthy donor.

### Example 2: Clinical Specimens

As described above, the validated 20-gene panel for variants associated with inherited hearing loss predisposition was applied to the molecular diagnosis of three unique de-identified patient specimens including a patient with Usher syndrome (Patient A), a known carrier (Patient B), and a healthy donor sample (Patient C). DNA sequencing libraries were made using Agilent's SureSelect XT HS2 kit and sequenced on an Illumina NGS platform (NextSeq 550Dx).

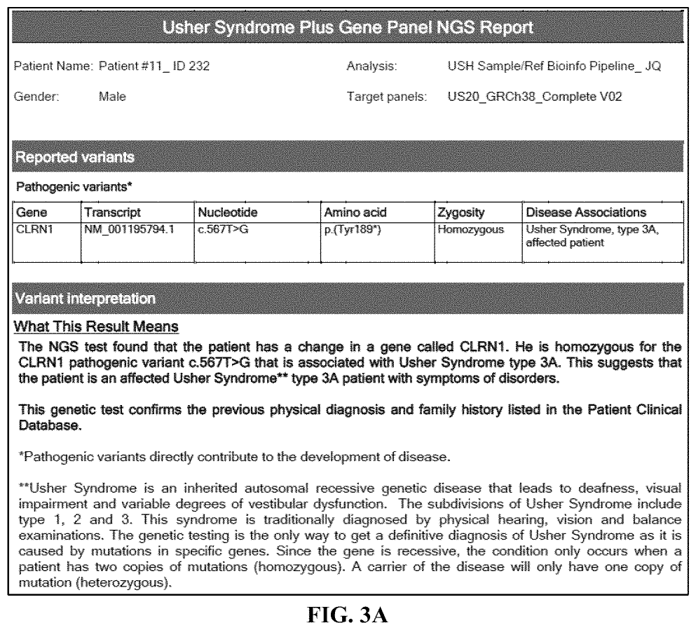

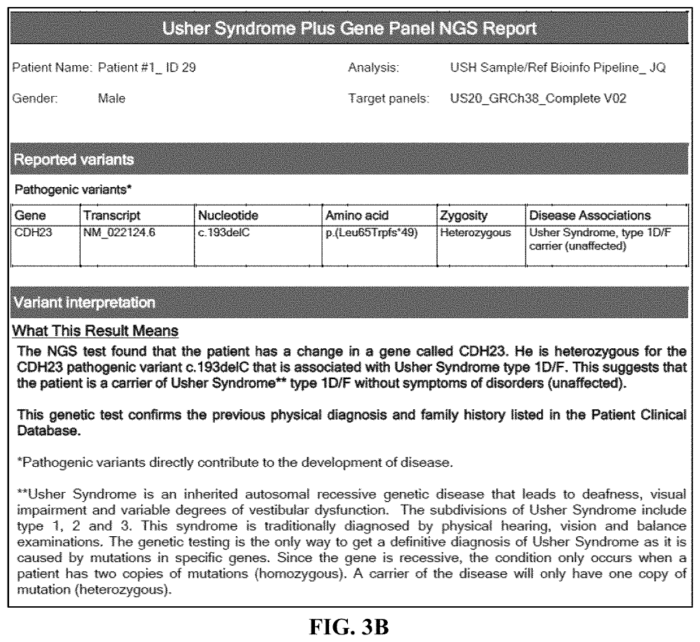

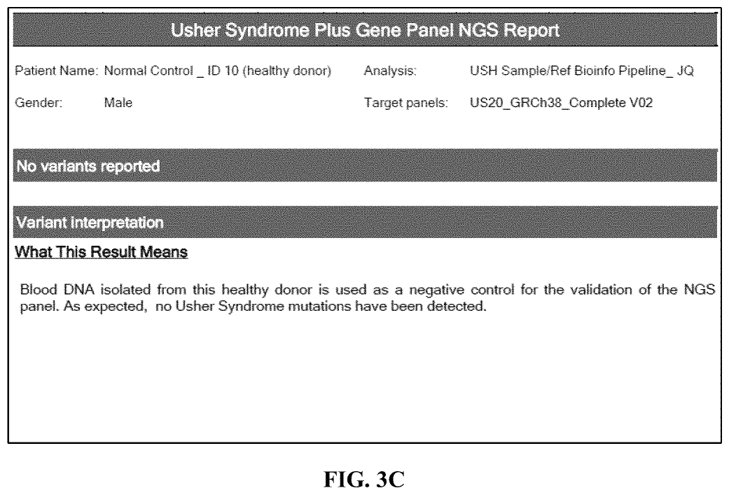

The methodology used for the molecular diagnosis of each patient is described in detail above. In summary, targeted DNA was enriched and sequenced using the in-house Usher Syndrome Plus NGS Test. NGS data were then analyzed to identify sequence variants involved in hearing loss, mainly due to Usher Syndrome. SNP and indel variants were then classified using the ACMG/AMP guidelines. For each Patient A-C below, the hybrid capture based NGS assay used 5437 probes to cover the coding region plus 10 bases of UTRs of the 20 genes shown in Table 1. The sequencing reads were trimmed and checked for quality using an in-house protocol described above (e.g, trimming FastQ to remove adaptor sequences, extracting dual molecular barcodes using AGeNT, followed by reads quality check using FastQC). These pre-processed data were then further analyzed and interpreted by an optimized bioinformatics workflow using the commercial Agilent's Alissa software tool. The results were then reported in a clinical setting format (see FIGS. 3A-3C showing representative reports of the Usher Syndrome Plus NGS Test).

### I. Identification of Homozygous CLRN1 Mutations in Affected Usher Syndrome Patient

FIG. 3A shows a representative report of the Usher Syndrome Plus NGS Test for a known Usher syndrome Type 3A, patient A (“Patient #11_ID 232, Male, “USH Sample/Ref Bioinfo Pipeline_JQ”). The test successfully identified an alteration/change in a gene called CLRN1 (transcript ID NM_001195794.1) and confirmed that the patient is homozygous for the CLRN1 pathogenic variant c.567T>G (Amino acid Tyr189) that is associated with Usher syndrome type 3A (FIG. 3A). The panel therefore confirmed the previous diagnosis and family history for Patient A.

Notably, the CLRN1 gene encodes a protein (clarin1) that contains a cytosolic N-terminus, multiple helical transmembrane domains, and an endoplasmic reticulum membrane retention signal, TKGH, in the C-terminus. The encoded protein may be important in development and homeostasis of the inner ear and retina. Mutations within this gene have been associated with Usher syndrome type IIIa. Multiple transcript variants encoding distinct isoforms have been identified for this gene. Clarin 1 protein is also active in the retina. Clarin 1 is likely to play a role in communication between nerve cells (neurons) in the inner ear and in the retina. Clarin 1 may also be important for the development and function of synapses.

### II. Identification of Heterozygous CDH23 Mutation in Usher Syndrome Carrier

FIG. 3B shows a representative report of the Usher Syndrome Plus NGS Test for known carrier of Usher syndrome, patient B (“Patient #1_ID29, Male, “USH Sample/Ref Bioinfo Pipeline_JQ”). A “carrier” is a patient known to have only one mutant gene for the disorder and who is therefore unaffected by the disorder. As shown in FIG. 3B, the Usher Syndrome Plus NGS Test successfully identified a change in a gene called CDH23 (transcript ID NM_022124.6) and confirmed that the patient is heterozygous for the CDH23 pathogenic variant c.193delC (Amino acid Leu65Trpfs49) that is associated with Usher syndrome type 1D/F (FIG. 3B). The panel therefore confirmed the previous diagnosis and family history for patient B.

Notably, the CDH23 gene is a member of the cadherin superfamily, whose genes encode calcium dependent cell-cell adhesion glycoproteins. The encoded protein is thought to be involved in stereocilia organization and hair bundle formation. The gene is located in a region containing the human deafness loci DFNB12 and USH1D. Usher syndrome 1D and nonsyndromic autosomal recessive deafness DFNB12 are caused by allelic mutations of this cadherin-like gene. Upregulation of this gene may also be associated with breast cancer. Alternative splice variants encoding different isoforms have been described.

### No Usher Syndrome Mutations Detected in Healthy Donor Sample

FIG. 3C shows a representative report of the Usher Syndrome Plus NGS Test for healthy donor, Patient C (“Normal Control_ID 10”, Male, “USH Sample/Ref Bioinfo Pipeline_JQ”). As shown in FIG. 3C, the Usher Syndrome Plus NGS Test confirmed that no Usher syndrome mutations were present for the healthy donor. Blood isolated from this healthy donor was used as a negative control for the validation of the 20-gene panel. The panel therefore confirmed the previous diagnosis and family history for Patient C.

### Example 3: Next Generation Sequencing and Bioinformatics Processing

Genomic DNA from whole blood or cultured cells was isolated. Isolated genomic DNA was mechanically sheared to an average size of between 100 and 300 bases. The fragmented DNA was then enzymatically repaired and end-modified with adenosine to make it receptive to T/A ligation with barcoded adaptors. The ligated products were size-selected, amplified, and then the regions of interest were captured using biotinylated RNA baits (SureSelect). In one example, baits were designed to cover the coding region plus 10 bases of UTRs of at least one of the 20 genes shown in Table 1. In another example, baits were designed to cover the coding region plus 10 bases of UTRs of all 20 of the genes shown in Table 1. In yet another example, baits were designed to capture all coding exons and exon/intron boundaries of the 20 hereditary hearing loss related genes listed in Table 1. Noncoding regions of these genes containing currently known pathogenic variants were also included. The DNA/RNA hybrids were enriched with streptavidin attached magnet beads (e.g., Dynabeads MyONe Streptavidin Tl, Thermo Fisher Scientific, Markham, ON) and subjected to washing under increasing stringency to remove non-targeted DNA sequences. A second amplification was performed (Agilent), followed by bead purification to remove all unused primers and nucleotides. In other embodiments, the libraries are purified post-PCR using AMPure beads.

The library is prepared as described above using Agilent's SureSelect XT HS2 kit and sequenced on an Illumina NGS platform (e.g. NextSeq 550Dx). The sequencing reads are trimmed and checked for quality using an in-house protocol. Specifically, the sequencing Fastq raw data were first trimmed to remove the adaptor sequences and to extract dual molecular barcodes using Agilent's Genomics NextGen Toolkit (AGeNT), followed by a reads quality check using the FastQC tool. These pre-processed data were further analyzed to create variant vcf files that were clinically interpreted by an optimized bioinformatics workflow using the commercial Agilent's Alissa software tool. The results were reported in a clinical setting format (see FIGS. 3A-C: three exemplar representative reports of the Usher Syndrome Plus NGS Testing).

Notably, the Agilent Genomics NextGen Toolkit (AGeNT) is a Java-based software module that processes read sequences from targeted high-throughput sequencing data generated by sequencing Agilent SureSelect and HaloPlex libraries. The Trimmer utility of the AGeNT module processes read sequences to identify and remove the adaptor sequences and extract dual molecular barcodes (for SureSelect XT HS2) as described above. The LocatIt utility of the AGeNT module processes the Molecular Barcode (MBC) information from HaloPlex HS, SureSelect XT HS, and SureSelect XT HS2 Illumina sequencing runs with options to either mark or merge duplicate reads and output in BAM file format.

Custom array probes for the 20 genes in the panel were designed using Agilent SureDesign custom design tool. Approximately 5,600 probes were designed. To design the probes, all 20 genes were inputted into the SureDesign software with the following parameters: hg 38 reference genome, RefSeq and Ensembl reference databases for gene definitions, including coding exons+5′ and 3′ UTRs with 10 bp flank. All of the regions that were not covered by the algorithm were evaluated and manually tiled across these regions with different combinations of probes using the highest stringency possible. This practice minimized off-target effects of regions that were harder to capture due to their lack of uniqueness in the human genome. All variants detected by the Usher Syndrome Plus NGS panel were manually reviewed by licensed personnel and classified by a team of variant scientists, according to the ACMG guidelines.

In some embodiments, enhanced assay specificity was achieved using long-range PCR. In one example, select exons from GJB2 and GJB6 were amplified from genomic DNA by long-range PCR. LR-PCR products were subjected to mechanical shearing, enzymatic end repair, and 3′ adenylation, followed by ligation to barcoded adaptors and a second PCR to enrich ligated fragments as described above. Final products from the LR-PCR library and the captured gDNA library were then combined and sequenced on an Illumina NextSeq instrument (NextSeq550 Dx). Following the sequencing reaction, sequence alignment and allele assignment were performed. BCL files from NextSeq550 were converted to FASTQ files. The raw sequence reads in Fastq files were then aligned to a custom reference genome. Reads were then sorted and indexed, followed by removal of read duplications. Average and minimum depth of coverage for every region of interest (ROI) were computed, and variant calling was performed. A variant call file (vcf) was then created and variant depth reports were created and loaded to a sequencing database. High-level annotation was then obtained for detected variants.

Annotation sources for all of the above examples included: 1. ClinVar (version 21) NCBI Clin Var 2020-12, 2. OMIM (version 16) OMIM 2021-01-06, 3. CGA (version 7) ClinGen CNV Atlas 2020-10-01, 4. COSMIC (version 18) COSMIC release v92, 5. Variant Function (version 39) Transcript based Variant annotation, 6. DGV (version 2) Database of Genomic Variants 2020-02-25, 7. ExAc (version 3) ExAC release 1.0—including GRCh38 from liftover data, 8. gnomAD (version 3) gnomAD release 2.0.2—with additional multi-allelic insertions and GRCh38 statistics from 1000 Genomes Phase 3 (version 2) 1000 Genomes Phase 3 release v5 (10 Sep. 2014) including GRCh38 dbSNP (version 6) dbSNP build 151, 9. ESP6500 (version 3) Variants in the ESP6500SI-V2 dataset of the exome sequencing project (ESP), annotated dbNSFP v3.0b2: Database of functional predictions for non-synonymous SNPs.

As described above, the 20-gene hearing loss predisposition panel disclosed herein demonstrated satisfactory performance for use in a clinical laboratory, with high sensitivity and specificity for SNVs (e.g., SNPs), small indels, and other variants. The panel can provide clinically significant information for hearing loss risk assessment and related risks including vision loss. The 20-gene hearing loss predisposition panel was shown to provide surprisingly increased levels of detection of pathogenic mutations compared to single-gene testing.

The aforementioned examples serve to illustrate embodiments of the present disclosure. These examples are in no way intended to limit the scope of the methods.

The methods illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof. It is recognized that various modifications are possible within the scope of the disclosure claimed. Thus, although the present disclosure has been specifically disclosed by preferred embodiments and optional features, modification and variation of the disclosure embodied therein herein disclosed may be resorted to by those skilled in the art, and that such modifications and variations are considered to be within the scope of this disclosure.

The disclosure being thus described, it will be obvious that the same may be varied in many ways. Such variations are not to be regarded as a departure from the spirit and scope of the disclosure and all such modifications are intended to be included within the scope of the following claims. The above specification provides a description of the manufacture and use of the disclosed compositions and methods. Since many embodiments can be made without departing from the spirit and scope of the disclosure, the disclosure resides in the claims.

What is claimed is:

1. A method for detecting at least one mutation in a plurality of hereditary hearing loss-related genes in a biological sample, the method comprising:
a) extracting genomic DNA from a biological sample obtained from a patient,
b) generating a library comprising a plurality of bait-captured gene sequences corresponding to a plurality of hereditary hearing loss-related genes, wherein the plurality of hereditary hearing loss-related genes comprises three or more of ABHD12, ADGRV1, ARSG, CDH23, CEP250, CEP78, CIB2, CLRN1, ESPN, FOX11, GJB2, GJB6, HARS2, KCNE1, KCNJ10, KCNQ1, MYO7A, PCDH15, PDZD7, SLC26A4, USH1C, USH1G, USH2A, and WHRN; and
c) detecting at least one mutation in at least one of the plurality of bait-captured gene sequences.

2. The method of claim 1, wherein the plurality of hereditary hearing loss-related genes comprises ABHD12, ADGRV1, ARSG, CDH23, CEP250, CEP78, CIB2, CLRN1, ESPN, FOX11, GJB2, GJB6, HARS2, KCNE1, KCNJ10, KCNQ1, MYO7A, PCDH15, PDZD7, SLC26A4, USH1C, USH1G, USH2A, and WHRN.

3. The method of claim 1, wherein the detecting step is performed using high throughput massive parallel sequencing.

4. The method of claim 1, wherein the biological sample is plasma, serum, or whole blood.

5. The method of claim 1, wherein the biological sample is a human biological sample from an infant patient, the infant patient having or suspected of having a hereditary hearing loss disorder.

6. The method of claim 1, wherein detecting at least one mutation in at least one of the plurality of bait-captured gene sequences indicates an increased susceptibility to hereditary hearing loss in the patient.

7. The method of claim 1, wherein an adaptor sequence is ligated to at least one end of the plurality of bait-captured gene sequences.

8. The method of claim 1, wherein an adaptor sequence is ligated to both ends of the plurality of bait-captured gene sequences.

9. A method for generating a library for the detection of at least one mutation in a plurality of hereditary hearing loss-related genes in a biological sample, wherein the library comprises a plurality of bait-captured gene sequences corresponding to the plurality of hereditary hearing loss-related genes, wherein the plurality of hereditary hearing loss-related genes comprises three or more of ABHD12, ADGRV1, ARSG, CDH23, CEP250, CEP78, CIB2, CLRN1, ESPN, FOX11, GJB2, GJB6, HARS2, KCNE1, KCNJ10, KCNQ1, MYO7A, PCDH15, PDZD7, SLC26A4, USH1C, USH1G, USH2A, and WHRN.

10. The method of claim 9, wherein the plurality of hereditary hearing loss-related genes comprises ABHD12, ADGRV1, ARSG, CDH23, CEP250, CEP78, CIB2, CLRN1, ESPN, FOX11, GJB2, GJB6, HARS2, KCNE1, KCNJ10, KCNQ1, MYO7A, PCDH15, PDZD7, SLC26A4, USH1C, USH1G, USH2A, and WHRN.

11. A method for detecting at least one mutation in a plurality of hereditary hearing loss-related genes in a biological sample, wherein the plurality of hereditary hearing loss-related genes comprises three or more of ABHD12, ADGRV1, ARSG, CDH23, CEP250, CEP78, CIB2, CLRN1, ESPN, FOX11, GJB2, GJB6, HARS2, KCNE1, KCNJ10, KCNQ1, MYO7A, PCDH15, PDZD7, SLC26A4, USH1C, USH1G, USH2A, and WHRN.

12. The method of claim 11, wherein the plurality of hereditary hearing loss-related genes comprises ABHD12, ADGRV1, ARSG, CDH23, CEP250, CEP78, CIB2, CLRN1, ESPN, FOX11, GJB2, GJB6, HARS2, KCNE1, KCNJ10, KCNQ1, MYO7A, PCDH15, PDZD7, SLC26A4, USH1C, USH1G, USH2A, and WHRN.

13. The method of claim 11, wherein the biological sample is obtained from an infant patient having or suspected of having a hereditary hearing loss disorder.

14. The method of claim 13, wherein the hereditary hearing loss disorder is Usher syndrome, Pendred syndrome, Jervell syndrome, or Lange-Nielsen syndrome.

15. The method of claim 11, wherein the biological sample is plasma, dried plasma, serum, dried serum, whole blood, or dried whole blood.

16. The method of claim 11, further comprising generating a library comprising a plurality of bait-captured gene sequences corresponding to each of the plurality of hereditary hearing loss-related genes.

17. The method of claim 11, wherein the at least one mutation in the plurality of hereditary hearing loss-related genes is detected using high throughput massive parallel sequencing.

18. The method of claim 11, the method further comprising the use of at least 5,000 nucleic acid probes.

19. The method of claim 18, wherein at least one of the at least 5,000 nucleic acid probes comprises a region of complementarity to at least one of the plurality of hereditary hearing loss-related genes, the region of complementarity comprising a coding region and 10 bases of an untranslated region (UTR) of the at least one of the plurality of hereditary hearing loss-related genes.

20. A kit for detecting at least one mutation in a plurality of hereditary hearing loss-related genes in a biological sample comprising a biosampling device and a lysis buffer, wherein the plurality of hereditary hearing loss-related genes comprises three or more of ABHD12, ADGRV1, ARSG, CDH23, CEP250, CEP78, CIB2, CLRN1, ESPN, FOX11, GJB2, GJB6, HARS2, KCNE1, KCNJ10, KCNQ1, MYO7A, PCDH15, PDZD7, SLC26A4, USH1C, USH1G, USH2A, and WHRN.

21. The kit of claim 20, further comprising one or more primer pairs that hybridize to one or more regions or exons of one or more of the plurality of hereditary hearing loss-related genes.

22. The kit of claim 20, further comprising one or more bait sequences that hybridize to one or more regions or exons of one or more of the plurality of hereditary hearing loss-related genes.
